(19) United States
(12) Patent Application Publication
(10) Pub. No.: US 2024/0298954 A1
(43) Pub. Date: Sep. 12, 2024

(54) Using Limited Lead Device EEG to Predict Delirium

(71) Applicant: UNIVERSITY OF SOUTH CAROLINA, COLUMBIA, SC (US)

(72) Inventors: MALISSA MULKEY, EASTOVER, SC (US); SUNGHAN KIM, GREENVILLE, NC (US); BAIJAIN YANG, WEST LAFAYETTE, IN (US)

(21) Appl. No.: 18/599,450

(22) Filed: Mar. 8, 2024

Related U.S. Application Data
(60) Provisional application No. 63/489,032, filed on Mar. 8, 2023.

(51) Int. Cl. A61B 5/372; A61B 5/00; A61B 5/256; A61B 5/291; G16H 10/60; G16H 50/20; G16H 50/70

(52) U.S. Cl. CPC A61B 5/372; A61B 5/256; A61B 5/291; A61B 5/6803; A61B 5/7207; A61B 5/7217; A61B 5/7275; G16H 10/60; G16H 50/20; G16H 50/70; A61B 2560/0431; A61B 2560/0468

(57) ABSTRACT

Method and system subject matter deals with predicting delirium in patients. As many as 80% of critically ill patients develop delirium increasing the need for institutionalization and resulting in higher morbidity and mortality. Clinicians detect less than 40% of delirium when using a validated screening tool. EEG is the criterion standard but is resource intensive thus not feasible for widespread delirium monitoring. This disclosure uses limited-lead rapid-response EEG and supervised deep learning methods with vision transformer to predict delirium. Vision transformer with rapid-response EEG is capable of predicting delirium. Such monitoring is feasible in critically ill older adults, for improving delirium detection accuracy, and providing greater opportunity for individualized interventions. Benefits may shorten hospital length of stay, increase discharge to home, decrease mortality, and reduce the financial burden associated with delirium.

### PRIORITY CLAIM

The present application claims the benefit of priority of U.S. Provisional Patent Application No. 63/489,032, titled Using EEG To Predict Delirium Using Limited Lead Device, filed Mar. 8, 2023, and which is fully incorporated herein by reference for all purposes.

### BACKGROUND OF THE PRESENTLY DISCLOSED SUBJECT MATTER

The disclosure concerns method and system subject matter dealing with predicting delirium in patients.

About 80% of ICU patients develop delirium, and about 60% of them will still have impaired memory and attention at discharge that prevents them from managing their treatment plan at home. Delirium triples one year mortality risk, and increases risk of need for long term care and hospital readmission costing ˜$164 billion/year. Delirium can occur in any setting or environment and is not limited to the ICU, hospital or healthcare facility. Older adults are at greater risk meaning the prevalence will increase as the population ages and more survive critical illness. Without an accurate method of detection, interventions and prevention strategies cannot accurately be tested. Bedside clinical assessments detect less than 20% of ICU delirium when a validated tool is used, and less than 10% of clinicians report using a validated method despite being a national guideline from more than 10 professional organizations for more than 10 years.

While the lack of an accurate method of detection prevents the use of interventions and prevention strategies, currently delirium is assessed using a bedside clinical screen that requires the patient be able to participate in the assessment. Thus, typically, such bedside clinical assessments are only able to detect less than 20% of ICU delirium even when a validated tool is applied.

Delirium is an acute syndrome manifested by changes in global cognitive function including either disorganized thinking or altered level of consciousness.[1] Delirium occurs in as many as 80% of critically ill older adults and is associated with worse long-term cognitive outcomes.[2, 3] For more than 20 years, at least 10 national and international professional organizations have included routine delirium screening in clinical practice guidelines.[4-6] Despite these recommendations and the availability of more than 40 validated screening tools, less than 10% of clinicians report routine delirium screening.[4, 7] Many critically ill patients are unable to participate in delirium screening, and are therefore untestable, due to coma or deep sedation.

Even with validated screening tools, delirium remains difficult to recognize, therefore, frequently underdiagnosed and undertreated. As duration and severity of delirium increases, it becomes increasingly more difficult to treat. Delirium is associated with a one-year increase in economic burden of more than$44K/patient, making it a global public health crisis.[8]

The electroencephalogram (EEG) is a representative signal describing the condition of the brain.[9, 10] The shape, amplitude, and oscillation speed help describe the condition and assist with diagnostics.[11, 12] Using EEG for delirium detection was first identified in the 1940's. Romano and Engel identified slowing of EEG with increases in sleep and decreases in awake waves when delirium was present.[13, 14] Thus, delirium has been reliably identified by changes in neural activity EEG. Unfortunately, the significant cost associated with technological set up and the need for expert analysis has prevented the use of EEG for delirium detection in the clinical environment.[15, 16]

More recently, user-friendly preprogrammed handheld EEG devices (as represented by FIG. 1) with recording accuracy equivalent to traditional EEG and rapid-response (rr) analytics such as machine learning have become available.[12, 17] As represented by FIG. 1, such types of devices offer rapid setup by anyone with limited training, providing rapid EEG data (thus rrEEG) within minutes, unlike traditional EEG that can take specially trained staff up to an hour to setup. To evaluate rrEEG waveforms, signal parameters are extracted and analyzed using computer based statistical algorithms. For example, nonlinear time series analysis offers insight into the dynamic nature and variability of brain signals.[18] With algorithms capable of accurate predictive detection, newer rrEEG devices may provide a feasible physiologic method to support delirium detection.

A widely used machine learning technique in research and practice is supervised machine learning where the ground truth is known to the researchers and labeled in the training dataset. Sophisticated deep neural networks are often adopted and optimized to fit the needs of specific learning tasks in real world scenarios. While the deep-learning-based approach often outperforms traditional statistical machine learning algorithms in prediction accuracy, its Blackbox nature often makes the model impractical to use in the medical field. Additional modules are often needed to help explain why or how the decision was made by deep learning algorithms.

### SUMMARY OF THE PRESENTLY DISCLOSED SUBJECT MATTER

Presently disclosed methodology and associated/related systems deal generally with predicting delirium in patients, and more particularly for some embodiments with using limited-lead rapid-response EEG and supervised deep learning methods with vision transformer to predict delirium.

Per presently disclosed subject matter, using a wireless EEG device using fewer leads than traditional EEG and a machine learning algorithm, delirium can be detected prior to patients having symptoms and with significantly greater accuracy and in patients that currently cannot be assessed. Further, such presently disclosed subject matter can advantageously be used in home, hospital, or long term care settings with little to no training.

Per some presently disclosed embodiments, use of a vision transformer with rapid-response EEG is capable of predicting delirium. Such monitoring is feasible in critically ill older adults, for improving delirium detection accuracy, and providing greater opportunity for individualized interventions. Benefits may shorten hospital length of stay, increase discharge to home, decrease mortality, and reduce the financial burden associated with delirium.

In accordance with presently disclosed subject matter, for some embodiments, using machine learning and limited lead EEG delirium can be detected prior to the onset of symptoms with 98% accuracy.

It is to be understood that the presently disclosed subject matter equally relates to both systems and to associated and/or corresponding methodologies. One exemplary such method relates to a method for predicting delirium in patients which integrates a deep learning based model with electroencephalogram (EEG) data, the method comprising training a machine-learned supervised deep learning method model to predict the presence of delirium in a patient based on training data associated with at least a plurality of limited-lead rapid-response EEG training data sets from patients; obtaining EEG test data associated with a target patient to be tested for the presence of delirium; inputting the EEG test data into the machine-learned supervised deep learning method model; and receiving, as output of the model, a positive or negative prediction of whether the target patient is experiencing the presence of delirium.

Another exemplary such method relates to a method for predicting delirium in patients which integrates a deep learning based model with electroencephalogram (EEG) data, the method comprising training a machine-learned supervised deep learning method vision transformer based model to predict the presence of delirium in a patient based on training data associated with at least a plurality of limited-lead rapid-response EEG training data sets from patients; obtaining EEG test data using a limited-lead rapid-response EEG device associated with a target patient to be tested for the presence of delirium; inputting the EEG test data into the machine-learned supervised deep learning method model; and receiving, as output of the model, a positive or negative prediction of whether the target patient is experiencing the presence of delirium.

Other example aspects of the present disclosure are directed to systems, apparatus, tangible, non-transitory computer-readable media, user interfaces, memory devices, and electronic devices for predicting delirium in patients. To implement methodology and technology herewith, one or more processors may be provided, programmed to perform the steps and functions as called for by the presently disclosed subject matter, as will be understood by those of ordinary skill in the art.

Another exemplary embodiment of presently disclosed subject matter relates to a system for predicting delirium in patients which integrates a deep learning based model with electroencephalogram (EEG) data, the system comprising a machine-learned supervised deep learning method model trained to predict the presence of delirium in a patient based on training data associated with at least a plurality of limited-lead rapid-response EEG training data sets from patients; one or more processors; and one or more non-transitory computer-readable media that store instructions that, when executed by the one or more processors, cause the one or more processors to perform operations. Such operations preferably comprise obtaining EEG test data associated with a target patient to be tested for the presence of delirium; inputting the EEG test data into the machine-learned supervised deep learning method model; and receiving, as output of the model, a positive or negative prediction of whether the target patient is experiencing the presence of delirium.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates examples of rapid-response EEG (rrEEG) headbands that circumscribe the head of a patient, and user-friendly preprogrammed handheld EEG devices, such as may be used in accordance with presently disclosed subject matter;

FIG. 2 diagrammatically illustrates exemplary model structure of a supervised deep learning model (Vision Transformer; ViT) used in a presently disclosed exemplary embodiment;

FIG. 3 graphically illustrates an example of a 30% overlapping data slice of 1 second (250 rows) in accordance with presently disclosed subject matter;

FIG. 4 illustrates in table form (Table 1) the results of cleaned data using individual component analysis (IDA) to remove noises, in accordance with presently disclosed subject matter;

FIG. 5 graphically illustrates the impact of data slices on prediction accuracy, by comparing test accuracy vs specific exemplary sizes of data slice; and

FIG. 6 graphically illustrates training convergence speed for exemplary embodiments of presently disclosed subject matter.

Repeat use of reference characters in the present specification and drawings is intended to represent the same or analogous features, elements, or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to methodology and associated/related systems dealing generally with predicting delirium in patients. More particularly, some embodiments are directed to using limited-lead rapid-response EEG and supervised deep learning methods with vision transformer to predict delirium.

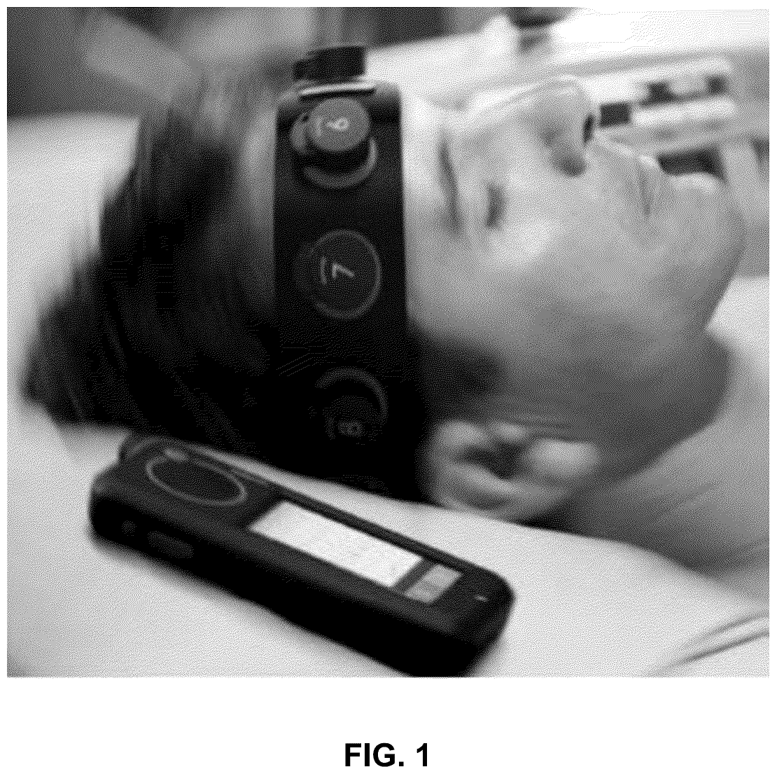

FIG. 1 represents a patient wearing an exemplary headband to be used such as with associated user-friendly preprogrammed handheld EEG devices for recording accuracy equivalent to traditional EEG. Rapid-response (rr) analytics such as machine learning can be used with such headband technology. Embodiments in accordance with presently disclosed technology and utilizing such features offer rapid setup by anyone with limited training, providing rapid EEG data (thus rrEEG) within minutes, much faster than setup time for more traditional EEG.

Per presently disclosed embodiments, to evaluate rrEEG waveforms, signal parameters may be extracted and analyzed using computer based statistical algorithms capable of accurate predictive detection, so that such presently disclosed rrEEG device embodiments may provide a feasible physiologic method to support delirium detection.

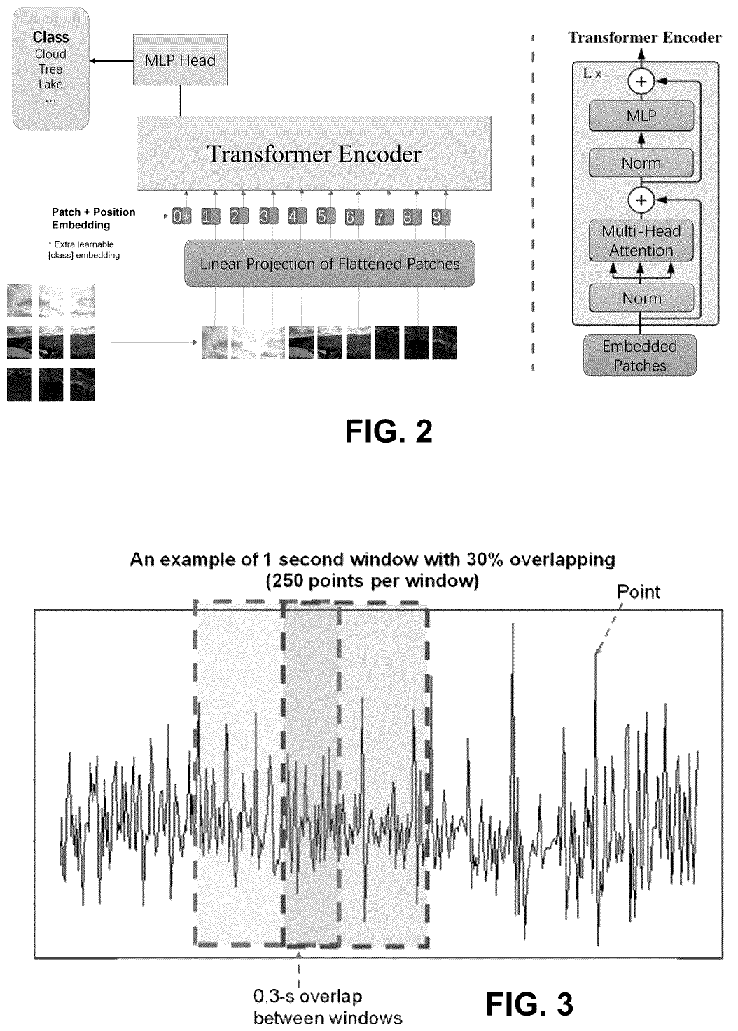

As otherwise noted herein, one known general machine learning technique in research and practice is referred to as supervised machine learning, where the ground truth is known to the researchers and labeled in the training dataset. For some exemplary embodiments disclosed herewith, a supervised deep learning model used is Vision Transformer (ViT).[19-21] FIG. 2 diagrammatically illustrates the model structure of such a Vision Transformer embodiment.

As represented in FIG. 2, the ViT model leverages cutting-edge transformer architecture that has revolutionized the field of natural language processing. To make the Transformer model work for image classification tasks, the core idea is to slice an image into a matrix of n×n sub-images. These sub-images are treated as sequential data so self-attention mechanisms can be applied to measure the relationship between pairs of sub-images. Stated another way, ViT (vision transformer) is a deep learning model that utilizes transformers which had been originally designed for natural language processing. They instead are now used for image recognition tasks. ViT breaks down an image into patches, processes them using such transformers, and then aggregates the information for classification or for object detection. Because ViT can maintain spatial and temporal information, it is ideal for analyzing sequential and spatial relationship in EEG. This disclosure describes how machine learning using ViT can serve as a billable low cost and low risk electronic means for detection of delirium.

### Materials & Methods

A proof-of-concept pilot approach for Methods of Detecting Neurological Delirium (MIND) uses a prospective design to evaluate use of supervised deep learning, ViT, and a rrEEG device for predicting delirium in critically ill older adults requiring mechanical ventilation. Such initial prospective proof-of-concept approach uses a rrEEG device providing data from all cerebral lobes, and the presently disclosed ViT method to evaluate rrEEG for the presence of delirium in critically ill patients. For such initial approach, written informed consent was obtained from the participant's legally authorized representative prior to any research activities, and all research procedures were conducted in accordance with the ethical standards set by the UMCIRB IRB and the Helsinki Declaration of 1975.

### Setting/Sample

The protocol for review of such approach was previously published.[22] In brief, seventeen patients meeting inclusion and exclusion criteria were recruited from three intensive care units (cardiac, medical, and surgical ICU) in a large rural academic medical center in North Carolina between March 2019 and March 2020. All participants 50 years old or older, required mechanical ventilation for greater than 12 hours, and were English speaking. Exclusion criteria included acute brain injury, seizures, or condition preventing participation in the delirium screening.

Each day, participants were assessed for the ability to participate in a delirium screening determined using the Richmond Agitation Sedation Scale (RASS).[23, 24] The RASS is a 10 level scale (+4 “combative” to −5 “unarousable”) with excellent inter-rater reliability (r=0.956, lower 90% confidence limit=0.948; k=0.73, 95% confidence interval=0.71, 0.75).[23, 24] A RASS score of −2 or higher (able to open eyes for >10 seconds to voice) met eligibility. Demographic and clinical characteristics were obtained from the electronic medical record.

### Measures/Label the Data

Bedside Behavioral Assessment for Delirium, The Confusion Assessment Method for the ICU (CAM-ICU), is a modified version of the CAM developed to assess mechanically ventilated and non-verbal patients.[25, 26] The CAM-ICU is based on the gold standard for delirium identification, the Diagnostic and Statistical Manual for Mental Disorder IV (DSM-IV) and one of two delirium screening tools recommended for use by the Society of Critical Care Medicine (SCCM).[1, 4, 27] The CAM-ICU has also been validated for use with older adults.[6, 28] Therefore, the CAM-ICU was considered the gold standard for determining delirium status. The CAM-ICU requires patient participation to identify four key delirium features including an a) acute onset or fluctuation in mental status within the previous 24 hours, (b) inattention, (c) altered level of consciousness [Richmond Agitation Sedation Scale (RASS) not=0], and (d) disorganized thinking.[25, 26] When used in research, the CAM-ICU has high sensitivity and specificity, 93 and 98% respectively and high inter-rater reliability at k.0.79.

### Physiologic Assessment for Delirium

Rapid-response EEG (rrEEG) headbands that circumscribe the head (as represented in FIG. 1) were applied to participants daily. Accuracy of placement is based on headband location (fastener in the center of the forehead, electrodes are numbered 1-10, and connects to the recorder at the hairline in the center on the back of the head). rrEEG waveforms are immediately visualized on the recorder and identifies the quality of the connection (e.g., impedance) using a color-coded diagram (green=low impedance/red=high impedance) of the headband. rrEEG monitoring occurred for 2 hours each evening between 1700-2100 for four days or ICU discharge. After one hour of rrEEG monitoring, the participant was assessed for delirium by the research team using the CAM-ICU (as noted above, considered herein as the gold standard for determining delirium status). To be considered delirium positive, the participant must have at least three of the core components of delirium including an acute change in baseline mental status, inattention, and either an altered level of consciousness or disorganized thinking.

FIG. 1 illustrates an example of such a rapid-response EEG (rrEEG) headband, as well as an example of a user-friendly preprogrammed handheld EEG device, both of which may be used in accordance with presently disclosed subject matter. During such use, the represented handheld device would be preprogrammed in accordance with presently disclosed subject matter re improved prediction of delirium in patients.

rrEEG Processing

rrEEG data were processed to remove artifacts such as muscle movement in the face and interference from nearby devices such as ventilators and cardiac monitors using high and low frequencies filters. Data are then re-referenced to estimate physiological noise and divided into multiple discrete time periods called epochs. rrEEG data is further “cleaned” using individual component analysis (IDA) to remove noises and generate features needed for machine learning algorithms. Component analysis is a widely accepted method to separate artifact from cortical processes data.[18, 29] The benefit of IDA with higher order statistics is the ability to simply subtract artifacts by directly examining the data's independent components.

### Machine Learning

EEG analytic techniques across studies have varied. Therefore, both traditional and three machine learning methods were used to analyze these data, specifically random forest (series of decision trees), stepwise linear discriminant analysis (removes variables that do not help classify the data, in this case delirium−/delirium+), and support vector machine (computer builds a model to provide the greatest difference between categories, in this case delirium−/delirium+). Due to challenges with feature selection, a supervised deep learning method, ViT, was primarily used.

Many deep learning applications, having sophisticated data processing techniques and feature engineering, are often not needed because the deep neural networks can learn those subtle features directly from the input data. Therefore, two types of data were studied. The first type of input data was preprocessed (muscle movement and device interference removal) and IDA cleaned, while the second type of input received only preprocessing with no IDA cleaning.

rrEEG devices extracted data every 4 ms, and each data sample contained readings from 10 sensors. A continuous number of rows of data are organized into a data slice, which is an 8×n array, where n represents the number of rows. These arrays are resized to 224×224 using bilinear interpolate and treated as images to feed into the ViT model. Note the diagrammatical representations of FIG. 2, as also otherwise referenced herein.

To understand how the results are related to the size of the slices, five different lengths were chosen: 25 rows (0.1 s), 125 rows (0.5 s), 250 rows (1 s), 400 rows (1.6 s), and 1250 rows (5 s). Due to the small sample size, data are augmented using an overlapping window scheme, where the starting row of the next data slice is located somewhere in the current data slice, rather than after the last row of the current data slice. An example of 30% overlapping data slice of 1 second (250 rows) is graphically represented in FIG. 3. Relevant signals in the rrEEG included the alpha (8-12 Hz), beta (15-30 Hz), delta (0.5-3 Hz), gamma (>30 Hz), and theta (4-7 Hz) waves. If we use ¬¬T to represent the time span of each data slice, then the frequency an image can detect is f=1/T. In conjunction with present initial embodiments of the presently disclosed subject matter, the highest frequency that can be detected is 10 Hz when T=0.1 s, and the lowest frequency it can detect is 0.2 Hz, when T=5 s. Data slices are randomly split into testing and training sets while avoiding putting all the data from any one subject into one (training+testing) set. Positive and negative cases in both the training sets and the testing sets are relatively balanced, with a ratio close to 1.

The following default hyper-parameters of the model were used in conjunction with present initial embodiments of the presently disclosed subject matter: batch size=64, learning rate=0.001, depth=12, and heads=8. Overlapping ratio of 75%, 90%, and 95% were reviewed. Since no major differences were discovered among these different overlapping ratios, 90% windows overlapping ratio was used to report the results. rrEEG data converges very quickly when using the ViT model. In most situations, training accuracies achieved more than 99% in as little as 3 epochs. To avoid overfitting, models trained after 5 epochs were used to evaluate the testing datasets.

To better understand the value of the ViT model in rrEEG data analyses, a public dataset Goldberger, Amaral[30] were also used to perform a binary classification task. The data slice of 1,250 rows was adopted and the overlapping ratio was set to 90%. The model achieved testing accuracy of 86.33%, which is better than state-of-the-art algorithms SleepEEGNet at the accuracy of 80.03%[31]. The initial review results show that ViT is a better fit to analyze rrEEG data than existing algorithms.

### Results

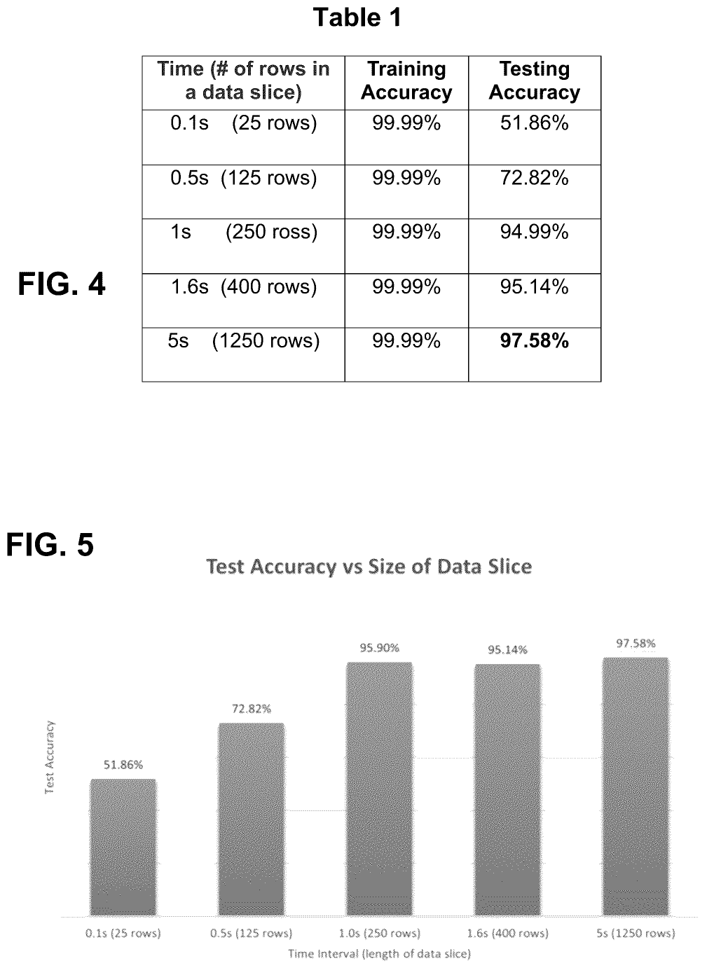

Fifteen different models (5 data slice sizes×3 overlapping rates) were used to evaluate the performance of the ViT models. Since overlapping rates did not impact the accuracy results, only findings of the overlapping rate=90% are reported, see FIG. 4 (Table 1), showing in table form the Results of IDA cleaned data (overlapping rate=90%). The model reached the best testing accuracy at 97.58% when the data slice was 1250 rows (5s).

FIG. 5 graphically illustrates the impact of data slices on prediction accuracy, by comparing test accuracy vs specific exemplary sizes of data slice. When the data slice is as little as 25 rows, it contains a full cycle of waves that are higher than 10 HZ. The prediction accuracy was merely 51.86%, suggesting the ViT model did not learn anything useful from the data. The bad results could suggest that waves with higher frequencies are less important when predicting delirium. It could also be because the sizes of the data sets were too small to be resized to 224×224 images.

When a data slice contains at least a full cycle of 2 Hz waves (0.5 second, or 125 rows), the accuracy increased to 72.82%. When a data slice contains at least a full cycle of 1 Hz waves (1 second, or 250 rows), the accuracy is further increased to around 95%. Better results are associated with lower frequency waves. Further review and testing would be needed to fully understand if long wave signals, such as delta waves, are truly the predictors for delirium.

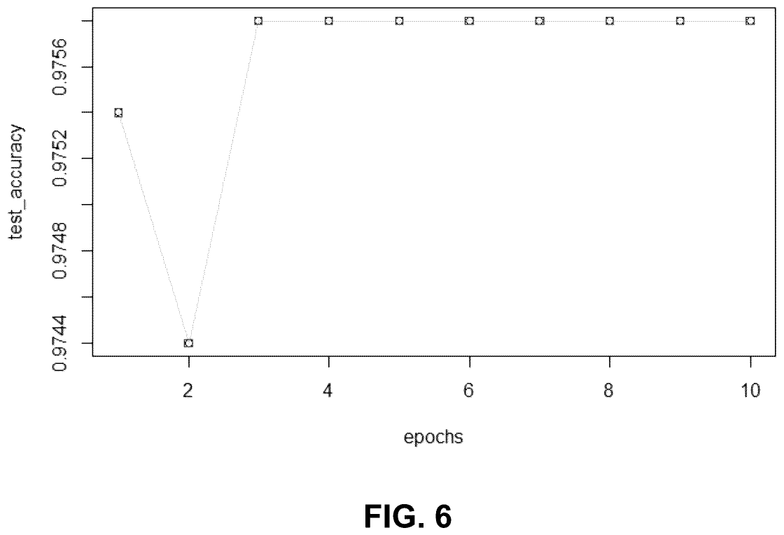

FIG. 6 graphically illustrates the training convergence speed. In most of the experiments, the models converged in 3 epochs. Accuracies did not improve from epoch 6 to epoch 10.

As for the training speed, under the setting of 1250 rows and 90% overlapping rate, about 2 hours were required. The training time is proportional to the size of training set. Recall that the data slice will be resized to 224×224, model with 250 s row, and 90% overlapping rate would take 10 hours to train.

Since ViT worked well on data cleaned using IDA, it begs the question: would ViT provide good prediction results on datasets not cleaned by IDA? To answer this question, uncleaned data were also fed into the ViT model to evaluate the results. Data slices of 1,250 were chosen with an overlapping rate of 90%. Both training and testing accuracies reached greater than 99.99%. The model converged within 3 epochs, as shown in FIG. 6. This suggests that deep learning techniques like ViT probably do not need additional feature engineering techniques to achieve great results. However, further studies could additionally validate such hypothesis.

The datasets were limited to only 12 subjects and used only on delirium predictions. One question could be what about other rrEEG datasets. We did another experiment to understand if ViT is still applicable by applying ViT to a public EEG data set[30] using a binary classification task. Under a window size of 1250 points, the model reached 86.33% test accuracy.

### Data Availability

The datasets generated and/or analyzed in conjunction with present initial embodiments of the presently disclosed subject matter are not publicly available but are available from the corresponding author on reasonable request.

### Discussion

This is the first prospective application of presently disclosed technology using a 10-electrode rrEEG device providing data from all lobes in the cerebrum, supervised deep learning, and ViT to evaluate rrEEG for the presence of delirium. This initial approach established that such monitoring is feasible in critically ill older adults across medical, surgical, and cardiac ICUs. One significant determination regarding the presently disclosed technology is that, using supervised deep learning and a ViT platform, patients were accurately classified as delirium positive or delirium negative based on identifiable characteristics in rrEEG, thus predicting the presence of delirium. These results were replicated using three methods of machine learning, including stepwise linear discriminant analysis, support vector machines, and supervised deep learning using ViT. Compared to prior studies using various machine learning and preprocessing methods, the ViT model using the hyperparameters mentioned above has provided greater accuracy.

For example, van Sleuwen et al. (2022) used a 3-channel limited lead device to measure physiologically based methods using the CAM-Severity Score[32]. To obtain three channels or waveforms, they used 6-second EEG strips obtained from a 4-electrode frontal montage. Using this montage on 252 delirious and 121 non-delirious patients, they obtained accuracies of 0.63-0.73 on the ROC curve, meaning that the model accurately predicted 63-70% of true positives for every possible decision threshold of the model. Similarly, Yamanashi, Malicoat[33] were able to obtain AUCs of 63-76% using a bispectral EEG with two channels. As a result, they recommended further studies may benefit from deep learning models such as one of the aspects used in present evaluations.

The merit of this handheld rrEEG is its objectiveness compared to currently available bedside screening methods. Additionally, this method does not require the use of a large EEG machine and specialized technicians for electrode placement that frequently limit large volume mass screening such as that needed to screen for delirium in ICUs. rrEEG is easy to use by busy hospital staff with minimal training. Use of pre-programmed algorithms, such as the one described here, limits the need for skilled interpretation required when using traditional EEG. Therefore, this method of detection is not limited by subjectivity and rationalizing of results associated with bedside screening.

While the ViT model used above has good performance, there are still opportunities for further improved prediction. For example, using a window of 10-60 seconds instead of the 1-5 second windows typically used in this type of analysis may have provided more data points. During the pre-processing phase, the presently disclosed technology used sequence extraction, meaning samples are obtained in a chronological sequential order and at a finite length. It is possible that constant correction or interpolation (removing data obtained from lead electrode that is bad) may provide a further cleaned data set for analysis.

### Limitations

Limitations of the present analysis of presently disclosed technology include the small sample of 13 participants with 7 experiencing delirium during the monitoring period as determined by the CAM-ICU. EEG changes occur prior to symptom onset and therefore, some of the participants may have had subsyndromal delirium detected using rrEEG that were not detected using bedside screening (CAM-ICU). Delirium assessments were conducted by the researcher rather than using clinician assessments providing stronger reliability of delirium status. Heterogeneity of the sample with varying etiologies and medication exposures could have resulted in some rrEEG changes being reflected more than others in a subset of patients, thus minimizing generalizability. Despite limitations, consistent results have been obtained across methods of analysis (frequency ratios, supervised learning, and deep learning) providing strength to the findings.

### CONCLUSION

In this analysis, we trained a ViT model to analyze rrEEG data under the constraint of a small sample and hence a limited amount of data. Despite the use of a sequence extraction method for preprocessing, 97% accuracy is significantly better than the 40% accuracy of clinician derived CAM-ICU assessments. Therefore, this method has strong potential for improving accuracy of delirium detection, providing greater opportunity to implement and evaluate individualized interventions when delirium is more amenable to treatment. Additionally, rrEEG is significantly cheaper than traditional EEG without losing the benefit of being able to charge for a billable service.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims. In any event, while certain embodiments of the disclosed subject matter have been described using specific terms, such description is for illustrative purposes only, and it is to be understood that changes and variations may be made without departing from the spirit or scope of the subject matter. Also, for purposes of the present disclosure, the terms “a” or “an” entity or object refers to one or more of such entity or object. Accordingly, the terms “a”, “an”, “one or more,” and “at least one” can be used interchangeably herein.

### REFERENCES

1. Guha, M. Diagnostic and statistical manual of mental disorders: Dsm-5. Ref. Rev. (2014).
2. Bashar, F. R. et al. Post-ICU psychological morbidity in very long ICU stay patients with ARDS and delirium. J. Critical Care 43, 88-94 (2018).
3. Mulkey, M. A., Hardin, S. R., Olson, D. M. & Munro, C. L. Pathophysiology review: seven neurotransmitters associated with delirium. Clin. Nurse Specialist 32, 195-211 (2018).
4. Devlin, J. W. et al. Clinical practice guidelines for the prevention and management of pain, agitation/sedation, delirium, immobility, and sleep disruption in adult patients in the ICU. Critical Care Medicine 46, c825-c873 (2018).
5. Killaspy, H., Baird, G., Bromham, N. & Bennett, A. Rehabilitation for adults with complex psychosis: summary of nice guidance. BMJ 372 (2021).
6. on Postoperative Delirium in Older Adults, A. G. S. E. P. American geriatrics society abstracted clinical practice guideline for postoperative delirium in older adults. J. Am. Geriatr. Soc. 63, 142-150 (2015).
7. Hunter, A., Crouch, B., Webster, N. & Platt, B. Delirium screening in the intensive care unit using emerging QEEG techniques: A pilot study. AIMS Neuroscience 7, 1 (2020).
8. Gou, R. Y. et al. One-year Medicare costs associated with delirium in older patients undergoing major elective surgery. JAMA surgery 156, 430-442 (2021).
9. Geraedts, V. et al. Machine learning for automated EEG-based biomarkers of cognitive impairment during deep brain stimulation screening in patients with Parkinson's disease. Clin. Neurophysiol. 132, 1041-1048 (2021).
10. de Araújo, B. E. S. et al. Clinical features, electroencephalogram, and biomarkers in pediatric sepsis-associated encephalopathy. Sci. Reports 12, 1-12 (2022).
11. Welte, T. M. et al. Quantitative EEG may predict weaning failure in ventilated patients on the neurological intensive care unit. Sci. Reports 12, 1-9 (2022).
12. Yamanashi, T. et al. Topological data analysis (TDA) enhances bispectral EEG (BSEEG) algorithm for detection of delirium. Sci. Reports 11, 1-9 (2021).
13. ENGEL, G. L. & ROMANO, J. Delirium: Ii. reversibility of the electroencephalogram with experimental procedures. Arch. Neurol. & Psychiatry 51, 378-392 (1944).
14. Romano, J. & Engel, G. L. Delirium: I. electroencephalographic data. Arch. Neurol. & Psychiatry 51, 356-377 (1944).
15. Van Der Kooi, A. W. et al. Delirium detection using EEG. Chest 147, 94-101 (2015).
16. Mulkey, M. A. et al. Rapid handheld continuous electroencephalogram (EEG) has the potential to detect delirium in older adults. Dimensions Critical Care Nurs. 41, 29-35 (2022).
17. Kamousi, B. et al. Comparing the quality of signals recorded with a rapid response ceg and conventional clinical EEG systems. Clin. Neurophysiol. Pract. 4, 69-75 (2019).
18. Kannathal, N., Acharya, U. R., Lim, C. M. & Sadasivan, P. Characterization of EEG-a comparative study. Comput. Methods Programs Biomed. 80, 17-23 (2005).
19. Dosovitskiy, A. et al. An image is worth 16×16 words: Transformers for image recognition at scale. arXiv preprint arXiv: 2010.11929 (2020).
20. Vaswani, A. et al. Attention is all you need. Adv. Neural Information Processing Systems 30 (2017).
21. Kostas, D., Aroca-Ouellette, S. & Rudzicz, F. Bendr: using transformers and a contrastive self-supervised learning task to learn from massive amounts of EEG data. Front. Hum. Neurosci. 253 (2021).
22. Mulkey, M. A. et al. Methods of identifying delirium: A research protocol. Res. Nursing & Health 42, 246-255 (2019).
23. Sessler, C. N. et al. The Richmond agitation-sedation scale: validity and reliability in adult intensive care unit patients. Am. Journal Respiratory Critical Care Medicine 166, 1338-1344 (2002).
24. Sessler, C. N. & Keane, K. Validity and reliability of a new agitation-sedation scale in a medical ICU population. Chest 118, 95S-95S (2000).
25. Ely, E. W. et al. Delirium in mechanically ventilated patients: validity and reliability of the confusion assessment method for the intensive care unit (CAM-ICU). JAMA 286, 2703-2710 (2001).
26. Ely, E. W. et al. Evaluation of delirium in critically ill patients: validation of the confusion assessment method for the intensive care unit (CAM-ICU). Critical Care Medicine 29, 1370-1379 (2001).
27. Diagnostic and statistical manual of mental disorders, (DSM-IV), Washington DC American psychiatric association, 1994. Tradução portuguesa. Fernandes J C edt, Climepsi Ed. Lisboa (1996).
28. National institute for health and care excellence: Clinical guidelines, in delirium: prevention, diagnosis and management. Natl. Inst. for Heal. Care Excell. (2019).
29. Delorme, A., Sejnowski, T. & Makeig, S. Enhanced detection of artifacts in EEG data using higher-order statistics and independent component analysis. Neuroimage 34, 1443-1449 (2007).
30. Goldberger, A. L. et al. Physiobank, physiotoolkit, and physionet: components of a new research resource for complex physiologic signals. Circulation 101, c215-c220 (2000).
31. Mousavi, S., Afghah, F. & Acharya, U. R. Sleepeegnet: Automated sleep stage scoring with sequence to sequence deep learning approach. PloS one 14, c0216456 (2019).
32. van Sleuwen, M. et al. Physiological assessment of delirium severity: the electroencephalographic confusion assessment method severity score (E-CAM-S). Critical Care Medicine 50, e11-e19 (2022).
33. Yamanashi, T. et al. Bispectral EEG (BSEEG) quantifying neuro-inflammation in mice induced by systemic inflammation: A potential mouse model of delirium. J. Psychiatric Research 133, 205-211 (2021).

What is claimed is:

1. A method for predicting delirium in patients which integrates a deep learning based model with electroencephalogram (EEG) data, the method comprising:
training a machine-learned supervised deep learning method model to predict the presence of delirium in a patient based on training data associated with at least a plurality of limited-lead rapid-response EEG training data sets from patients;
obtaining EEG test data associated with a target patient to be tested for the presence of delirium;
inputting the EEG test data into the machine-learned supervised deep learning method model; and
receiving, as output of the model, a positive or negative prediction of whether the target patient is experiencing the presence of delirium.

2. The method according to claim 1, wherein the EEG test data is obtained using a limited-lead rapid-response EEG device.

3. The method according to claim 1, wherein the supervised deep learning method is vision transformer based.

4. The method according to claim 3, wherein the supervised deep learning method is trained using ground truth of delirium corresponding to the plurality of limited-lead rapid-response EEG training data sets from patients.

5. The method according to claim 1, wherein the EEG training data sets are derived from patients who are critically ill older adults.

6. The method according to claim 1, wherein obtaining and inputting the EEG test data and receiving the model output is conducted by a user operating a user-friendly preprogrammed handheld EEG device with rapid-response (rr) analytics.

7. The method according to claim 3, wherein the vision transformer based model operates by slicing an image into a matrix of n×n sub-images, processing the sub-images as sequential data to measure the relationship between pairs of sub-images, and then aggregating the relationship information for classification or for object detection, for analyzing sequential and spatial relationships in the EEG-based data.

8. The method according to claim 2, wherein the limited-lead rapid-response EEG device includes a headband with a plurality of electrodes that circumscribe the head of a target patient.

9. The method according to claim 1, wherein the EEG test data is processed for inputting into the machine-learned supervised deep learning method model by removing artifact signals from the EEG test data.

10. The method according to claim 9, wherein the EEG test data are filtered using high and low frequencies filters to remove artifacts from movement of the target patient or interference from nearby medical devices, and the EEG test data are then divided into multiple discrete time epochs for inputting into the machine-learned supervised deep learning method model.

11. A system for predicting delirium in patients which integrates a deep learning based model with electroencephalogram (EEG) data, the system comprising:
a machine-learned supervised deep learning method model trained to predict the presence of delirium in a patient based on training data associated with at least a plurality of limited-lead rapid-response EEG training data sets from patients;
one or more processors; and
one or more non-transitory computer-readable media that store instructions that, when executed by the one or more processors, cause the one or more processors to perform operations, the operations comprising:
obtaining EEG test data associated with a target patient to be tested for the presence of delirium;
inputting the EEG test data into the machine-learned supervised deep learning method model; and
receiving, as output of the model, a positive or negative prediction of whether the target patient is experiencing the presence of delirium.

12. The system according to claim 11, further comprising a limited-lead rapid-response EEG device for obtaining the EEG test data.

13. The system according to claim 11, wherein the supervised deep learning method is vision transformer based.

14. The system according to claim 13, wherein the supervised deep learning method is trained using ground truth of delirium corresponding to the plurality of limited-lead rapid-response EEG training data sets from patients.

15. The system according to claim 11, wherein the EEG training data sets are derived from patients who are critically ill older adults.

16. The system according to claim 11, further comprising a user-operated user-friendly preprogrammed handheld EEG device with rapid-response (rr) analytics, for control of operations of obtaining and inputting the EEG test data and receiving the model output.

17. The system according to claim 13, wherein the vision transformer based model includes slicing an image into a matrix of n×n sub-images, processing the sub-images as sequential data to measure the relationship between pairs of sub-images, and then aggregating the relationship information for classification or for object detection, for analyzing sequential and spatial relationships in the EEG-based data.

18. The system according to claim 12, wherein the limited-lead rapid-response EEG device includes a headband with a plurality of electrodes that circumscribe the head of a target patient.

19. The system according to claim 11, wherein operations further comprise the EEG test data being processed for inputting into the machine-learned supervised deep learning method model by removing artifact signals from the EEG test data.

20. The system according to claim 19, wherein operations further comprise the EEG test data being filtered using high and low frequencies filters to remove artifacts from movement of the target patient or interference from nearby medical devices, and the EEG test data are then divided into multiple discrete time epochs for inputting into the machine-learned supervised deep learning method model.

21. A method for predicting delirium in patients which integrates a deep learning based model with electroencephalogram (EEG) data, the method comprising:
training a machine-learned supervised deep learning method vision transformer based model to predict the presence of delirium in a patient based on training data associated with at least a plurality of limited-lead rapid-response EEG training data sets from patients;
obtaining EEG test data using a limited-lead rapid-response EEG device associated with a target patient to be tested for the presence of delirium;
inputting the EEG test data into the machine-learned supervised deep learning method model; and
receiving, as output of the model, a positive or negative prediction of whether the target patient is experiencing the presence of delirium.

22. The method according to claim 21, wherein:
the limited-lead rapid-response EEG device comprises a headband with a plurality of electrodes that circumscribe the head of a target patient; and
obtaining and inputting the EEG test data and receiving the model output is conducted by a user operating a user-friendly preprogrammed handheld EEG device with rapid-response (rr) analytics.

23. The method according to claim 21, wherein:
the vision transformer based model operates by slicing an image into a matrix of n×n sub-images, processing the sub-images as sequential data to measure the relationship between pairs of sub-images, and then aggregating the relationship information for classification or for object detection, for analyzing sequential and spatial relationships in the EEG-based data; and
the EEG test data are filtered using high and low frequencies filters to remove artifacts from movement of the target patient or interference from nearby medical devices, and the EEG test data are then divided into multiple discrete time epochs for inputting into the machine-learned supervised deep learning method vision transformer based model.
